(19) United States
(12) Patent Application Publication
(10) Pub. No.: US 2015/0118173 A1
(43) Pub. Date: Apr. 30, 2015

(54) FORMULATION COMPRISING CAPRYLIC ACID ETHANOLAMIDE AND/OR CAPRIC ACID ETHANOLAMIDE IN COMBINATION WITH AN ALUMINIUM SALT AND ITS USE

(71) Applicant: EVONIK INDUSTRIES AG, Essen (DE)

(72) Inventors: Mike Farwick, Essen (DE); Oliver Springer, Wesel (DE); Matthias Mentel, Dortmund (DE); Stefan Bergfried, Essen (DE)

(73) Assignee: EVONIK INDUSTRIES AG, Essen (DE)

(21) Appl. No.: 14/397,379

(22) PCT Filed: Mar. 28, 2013
(86) PCT No.: PCT/EP2013/056670

§ 371 Date: Oct. 27, 2014

(30) Foreign Application Priority Data
Apr. 27, 2012 (DE) 102012206999.2

(51) Int. Cl. A61Q 17/00; A61Q 5/00; A61Q 15/00; A61Q 19/00; A61K 8/26; A61K 8/42

(52) U.S. Cl. CPC A61Q 17/005; A61K 8/26; A61K 8/42; A61Q 15/00; A61Q 19/00; A61Q 5/006; A61K 2800/5922
USPC 424/68; 514/625; 424/682; 514/492

(57) ABSTRACT

The invention relates to a formulation comprising caprylic acid ethanolamide and/or capric acid ethanolamide in combination with an aluminium salt and to its use.

### FIELD OF THE INVENTION

The invention relates to a formulation comprising caprylic acid ethanolamide and/or capric acid ethanolamide in combination with an aluminium salt and to its use.

### PRIOR ART

Antimicrobial active ingredients are used widely in cosmetic deodorants or antiperspirants, antidandruff and antiacne formulations, footcare and intimate hygiene compositions and also oral hygiene and dental care products.

Body odour is formed primarily when the initially odourless perspiration is decomposed by microorganisms on the skin. Only the microbial degradation products cause the unpleasant sweat odour. This arises in particular wherever there is a high density of sweat glands and also a high density of odour-producing microbes, such as for example in the armpits, in intimate areas or on the feet. The microbes responsible for this, however, often do not belong exclusively to the genus Corynebacterium.

Our natural skin flora includes many different bacteria, including staphylococci, certain streptococci, occasionally enterococci, corynebacteria and propionibacteria. They live on the skin, sometimes in the pores, in sweat glands and sweat ducts, and also in the oral cavity. A variety of fungi also occur on the skin.

Certain skin disorders are also connected with excessive growth of undesired microorganisms on the skin. Thus, acne inter alia is caused by uncontrolled multiplication of the anaerobic skin bacterium Propionibacterium acnes. Candida albicans is responsible for the formation of thrush, dandruff is associated inter alia with the fungus Pilz Malassezia furfur.

In the area of oral hygiene, microorganisms play an essential role e.g. in the formation of caries and plaque. The most important cause of dental caries is for example Streptococcus mutans.

The most important strategies for controlling body odour are odour masking (by means of fragrances), odour absorption, control of the microorganisms involved in odour formation by means of antimicrobial substances, and the influencing of the sebaceous gland activity by means of antiperspirants. Of particular importance here are antiperspirants containing aluminium chlorohydrate (ACH), although these have the disadvantage that they leave behind traces of use on clothing.

WO2011032924 describes the use of certain amides including caproic acid isopropanolamide, caprylic acid isopropanolamide, capric acid isopropanolamide, lauric acid isopropanolamide, caprylic acid monoethanolamide, caprylic acid diethanolamide and caprylic acid N-methylethanolamide for reducing the growth of microorganisms and as a constituent of cosmetic formulations and proposes, among many other compounds which can additionally be co-used, also aluminium salts such as aluminium chlorohydrate, aluminium zirconium tetrachlorohydrex GLY, aluminium zirconium pentachlorohydrate and alum.

It was an object of the invention to provide an active ingredient combination which has excellent properties as regards its antimicrobial effect, in particular on the skin of living beings and thus in particular for use in cosmetic applications.

### DESCRIPTION OF THE INVENTION

Surprisingly, it has been found that the combination described below of caprylic acid ethanolamide and/or capric acid ethanolamide (C8- or 10-MEA) with ACH has a synergistic effect in deodorant applications.

The present invention therefore provides formulations comprising at least one compound of the general formula (I)

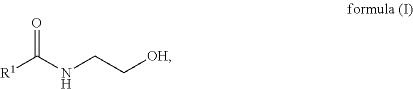

where

### R¹=—C₇H₁₅ or —C₉H₁₉

and an aluminium salt.

The invention further provides the use of the formulation according to the invention for reducing the growth of microorganisms, in particular on a surface, and also for reducing human body odour.

It is an advantage of the present invention that the formulations are stable in nonpolar and polar (in particular alcoholic or aqueous) systems since no hydrolysis or alcoholysis take place under the required conditions.

It is also an advantage that the effectiveness is largely constant over the entire pH range of the possible cosmetic formulations.

It is a further advantage that the formulations have good skin compatibility.

It is another advantage that they contribute to reducing the ACH content in antiperspirants.

The use according to the invention, in cases where it is a use on a living species, is exclusively a cosmetic and a non-therapeutic use.

Unless stated otherwise, all of the stated percentages (%) are percentages by mass. In connection with the present invention, the term “aluminium chlorohydrate” is understood as meaning the salts of the general empirical formula AlₙCl₍₃ₙ₋ₘ₎(OH)ₘ, in particular Al₂Cl(OH)₅.

Preference is given in particular to formulations according to the invention which are characterized in that the aluminium salt is selected from the group comprising, preferably consisting of:

aluminium acetate, aluminium behenate, aluminium benzoate, aluminium bromohydrate, aluminium butoxide, aluminium calcium sodium silicate, aluminium caprylate, aluminium capryloyl hydrolyzed collagen, aluminium chloride, aluminium chlorohydrate, aluminium chlorohydrex, aluminium chlorohydrex PEG, aluminium chlorohydrex PG, aluminium citrate, aluminium diacetate, aluminium dibenzoate/stearate hydroxide, aluminium dicetyl phosphate, aluminium dichlorohydrate, aluminium dichlorohydrex PEG, aluminium dichlorohydrex PG, aluminium dilinoleate, aluminium dimyristate, aluminium distearate, aluminium glycinate, aluminium hydrogenated tallow glutamate, aluminium hydroxy bis-methylene bis-di-t-butylphenyl phosphate, aluminium iron calcium magnesium germanium silicates, aluminium iron calcium magnesium zirconium silicates, aluminium iron silicates, aluminium isopropoxide, aluminium isostearate, aluminium isostearates/laurates/palmitates, aluminium isostearates/laurates/stearates, aluminium isostearates/myristates, aluminium isostearates/palmitates, aluminium isostearates/stearates, aluminium isostearyl glyceryl phosphate, aluminium laccate, aluminium lactate, aluminium lanolate, aluminium/magnesium hydroxide stearate, aluminium magnesium oxide, aluminium methionate, aluminium myristate, aluminium myristates/palmitates, aluminium PCA, aluminium phenolsulphonate, aluminium sesquichlorohydrate, aluminium sesquichlorohydrex PEG, aluminium sesquichlorohydrex PG, aluminium starch octenylsuccinate, aluminium stearate, aluminium stearates, aluminium stearoyl glutamate, aluminium sucrose octasulphate, aluminium sulphate, aluminium triformate, aluminium triphosphate, aluminium tristearate, aluminium undecylenoyl collagen amino acids, aluminium zinc oxide, aluminium zirconium trichlorohydrate, aluminium zirconium trichlorohydrex GLY, aluminium zirconium tetrachlorohydrate, aluminium zirconium tetrachlorohydrex GLY, aluminium zirconium tetrachlorohydrex PEG, aluminium zirconium tetrachlorohydrex PG, aluminium zirconium pentachlorohydrate, aluminium zirconium pentachlorohydrex GLY, aluminium zirconium octachlorohydrate, aluminium zirconium octachlorohydrex GLY, ammonium alum, ammonium silver zinc aluminium silicate, calcium aluminium borosilicate, cobalt aluminium oxide, magnesium/aluminium/hydroxide/carbonate, magnesium aluminium silicate, magnesium/aluminium/zinc/hydroxide/carbonate, potassium alum, potassium aluminium polyacrylate, silver magnesium aluminium phosphate, sodium alum, sodium aluminate, sodium aluminium chlorohydroxy lactate, sodium/aluminium hydroxide/oxalate/sulphate, sodium/aluminium/iron hydroxide/oxalate/sulphate, sodium/aluminium/iron/sulphate/citrate/hydroxide, sodium/aluminium/iron/sulphate/oxalate/hydroxide, sodium/aluminium/iron/sulphate/tartarate/hydroxide, sodium aluminium lactate, sodium phosphorus/zinc/calcium/silicon/aluminium/silver oxides, sodium potassium aluminium silicate, sodium silicoaluminate, sodium silver aluminium silicate, tromethamine magnesium aluminium silicate and alum, in particular aluminium chlorohydrate.

Preference is given to formulations according to the invention which are cosmetic or pharmaceutical formulations.

Cosmetic or pharmaceutical formulations according to the invention can be used as skincare, face care, head care, bodycare, intimate care, footcare, haircare, nailcare, dental care, lip care or oral care product or antiperspirant/deodorant product. Examples of haircare products are hair washing compositions, hair treatments, hair conditioners, hair fluid, hair gel, hair tonic, hair wax, hair lacquer, hairspray, hair cream, hair mousse, hair balm, antidandruff shampoo. Examples of bodycare products are shower bath, cream bath, cream gel, shower oil, shower gel, washing gel, washing scrub, cleansing lotion, face mask, facial toner, facial scrub, eye cream, night cream, cleansing mask, lotion pads, cleansing wipes, cleansing lotion, cleansing milk, cleansing gel, aftershave gel, aftershave balm, sun milk, aftersun products, self-tanning compositions, foot lotion, foot spray, body lotion, body gel, body spray, body milk, body scrub, body oil, body butter.

Examples of lipcare products are lip balm, lip cream, lipcare stick.

The cosmetic or pharmaceutical formulations according to the invention can contain e.g. at least one additional component, selected from the group of

emollients,
emulsifiers,
thickeners/viscosity regulators/stabilizers,
UV light protection filters,
antioxidants,
hydrotropes (or polyols),
solids and fillers,
film formers,
pearlescent additives,
deodorant and antiperspirant active ingredients,
insect repellents,
self-tanning agents,
preservatives,
conditioners,
perfumes,
dyes,
odour absorbers,
cosmetic active ingredients,
care additives,
superfatting agents,
solvents.

Substances which can be used as exemplary representatives of the individual groups are known to the person skilled in the art and can be found for example in the German application DE 102008001788.4. This patent application is hereby incorporated by reference and thus forms part of the disclosure.

For cases of using the formulation according to the invention on inanimate surfaces, said formulations are in particular cleaning and care compositions for domestic, industrial and institutional applications such as, for example, disinfectants, disinfectant cleaners, foam cleaners, floor cleaners, carpet cleaners, upholstery cleaners, floor care products, marble cleaners, parquet cleaners, stone and ceramic floor cleaners, wipe-care products, stainless steel cleaners, glass cleaners, dishwashing detergents, plastic cleaners, sanitary cleaners, wood cleaners, leather cleaners, laundry detergents, laundry care compositions, disinfectant detergents, heavy-duty detergents, gentle detergents, wool detergents, fabric conditioners, impregnation compositions.

It is obvious that mixtures of compounds of the general formula (I) can be present in formulations according to the invention. It is advantageous if such a formulation is characterized in that mixtures of compounds are present in which the weight ratio of compounds of the general formula (I) with an R₁=—C₇H₁₅ to compounds of the general formula (I) with an R₁=—C₉H₁₉ is in a range from 5:95 to 95:5, particularly preferably from 35:65 to 65:35.

However, it has been found that the presence of a high fraction of compounds similar to the compounds of the general formula (I), characterized by an R₁ with a chain other than —C₇H₁₅ or —C₉H₁₉ has a negative influence.

Consequently, preferred formulations according to the invention are characterized in that, in the formulation, less than 80% by weight, preferably less than 40% by weight, in particular less than 20% by weight, of compounds of the general formula (II)

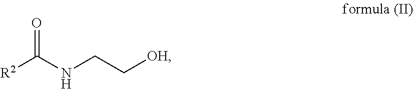

where
R²=organic radical with a chain length of less than 7 or greater than 9, based on the total weight of the compounds of the general formula (I) and (II) are present.

In this connection, R₁ and R₂ of the general formula (I) and (II) are independent of one another, R₁ and R₂ in the general formula (I) and (II) preferably being identical.

Preferably formulations according to the invention are in particular characterized in that they comprise 0.001% by weight to 20% by weight, preferably 0.01% by weight to 10% by weight, particularly preferably 0.1% by weight to 5% by weight, of at least one compound of the general formula (I), based on the total formulation.

Formulations according to the invention are in particular characterized in that they comprise 0.01 to 50% by weight, preferably 0.1% by weight to 35% by weight, particularly preferably 1.0% by weight to 20% by weight, of at least one aluminium salt, based on the total formulation.

The formulations according to the invention can be used as a multitude of formulations for applications in the home, industry, pharmacy and cosmetics. They are particularly suitable as effective components in deodorants, which may be present in the form of aerosol sprays, pump sprays, roll-on formulations, deodorant sticks, W/O or O/W emulsions (e.g. creams or lotions) or wipes. The active ingredients/active ingredient combinations known from the prior art can be co-used in these formulations, such as e.g. triclosan, ethylhexylglycerol, farnesol, polyglycerol caprate or caprylate, triethyl citrate, penta(carboxymethyl)diethylenetriamine (pentetic acid), pentylene glycol, propylene glycol, ethanol, zinc ricinoleate, cyclodextrins, silver and its salts or zinc oxide.

However, application is not limited to use in deodorants, but may be advantageous wherever it is desirable to control microorganisms or their growth, such as e.g. in intimate hygiene articles, antiacne or antidandruff products, which can be present in the form of customary leave-on or rinse-off formulations, such as e.g. creams, lotions, shampoos, washing solutions, hair conditioners, wipes and similar formulations. Use of the formulations according to the invention in footcare compositions is also advantageous; footcare compositions are understood as meaning not only compositions that are to be applied directly to the skin, but the term likewise encompasses for example deodorizing shoe insoles.

For antiacne products, the formulations according to the invention can optionally also comprise known antiacne active ingredients, such as e.g. dibenzoyl peroxide, salicylic acid, phytosphingosine, tretinoin, isotretinoin or plant extracts. Similarly, in the case of antidandruff products, combinations with known antidandruff active ingredients, such as e.g. climbazole, zinc pyrethione, selenium compounds (for example selenium sulphide), piroctone olamine (octopirox) or plant extracts, can be used.

The formulations according to the invention can also be used in the sector of oral hygiene products, in which case use in mouthwash solutions or toothpastes is recommended in particular.

They can also be used in combination with other active ingredients which have effectiveness gaps in areas in which the formulations according to the invention are effective for said purpose. Thus, the use of preservatives can be reduced or in some cases it is even possible to completely dispense with classical preservatives.

The invention further provides the use of at least one of the formulations according to the invention for reducing the growth of microorganisms.

As a result of the use according to the invention of the formulations according to the invention, it is possible for example to reduce the number of microorganisms in the formulation itself. This corresponds to the basic concept of a preservative.

However, the use according to the invention is preferably carried out on a surface. Preferably, the surface in the use according to the invention is the surface of a human or animal body part, in particular the skin, the hair or the teeth, with use on the skin being particularly preferred.

These surfaces generally have undesired microorganisms such as, for example, bacteria, specifically Corynebacterium xerosis, Corynebacterium jeikeium, Corynebacterium minutissimum, Propionibacterium acnes, Staphylococcus aureus, Staphylococcus epidermidis, Streptococcus sobrinus and Streptococcus mutans. Fungi (including yeasts) likewise also occur here, thus for example representatives of the genera Microsporum, Epidermiphyton, Trichphyton, and also Candida albicans or Malassezia furfur.

Consequently, a use characterized in that the microorganisms are Gram-positive, in particular coryneform, bacteria, and in particular likewise the aforementioned species.

In a further preferred embodiment of the use according to the invention, the surface on which the use takes place is the surface of an inanimate object. Examples of such surfaces are surfaces of tiles, wood, glass, ceramic, linoleum, plastic, painted surfaces, leather, materials, fibres. Preferably, such objects come into daily contact several times directly or indirectly through people or animals. Preferably, the surface of such objects experiences soiling as a result of the aforementioned contact. Examples of such objects are

window panes and windowsills, shower partitions,
floorings such as carpets, tiles, laminates, parquet, cork floorings, marble, stone and fine stoneware floors
household ceramics such as toilets, wash basins, bidets, shower trays, bath tubs, door handles, fittings,
domestic appliances such as washing machines, dryers, dishwashers, sinks made of ceramic or stainless steel, furniture such as tables, chairs, benches, work surfaces,
windows, pots and pans, crockery and cutlery,
laundry, in particular laundry worn close to the body (“underwear”)
watercraft, vehicles and aircraft, such as cars, buses, motorboats and sail boats
tools such as surgical instruments, vacuum cleaners.

Engines, pipelines, tanks and devices for transportation, processing and storage in food processing.

Consequently, in this connection, it is the use of the compounds of the general formula (I) in cleaning and care compositions for domestic, industrial and institutional trade.

The invention also further provides the use of at least one formulation according to the invention for reducing human body odour.

The invention also further provides the use of at least one formulation according to the invention for reducing the whitening effect. As a comparison formulation to which the reduced whitening effect refers, a formulation based only on the aluminium salt is used in particular which, in quantitative terms, has the same sweat-odour-inhibiting effect as the whitening-effect-reduced formulation.

Among the uses according to the invention, in each case preference is given to using those formulations which are specified above as preferred formulations.

The invention further provides a method for reducing the growth of microorganisms, involving the steps

a) provision of a formulation according to the invention, in particular of a cosmetic or pharmaceutical formulation,
b) application of the formulation to the surface to be treated, in particular skin, hair or teeth, in an effective amount,
c) leaving the formulation on the surface to be treated for a time which suffices to ensure the reduction in the growth of the microorganisms and
d) optionally washing out or rinsing off the formulation.

The examples given below describe the present invention by way of example without intending to limit the invention, the scope of application of which arises from the entire description and the claims, to the embodiments specified in the examples. The following figures form part of the examples.

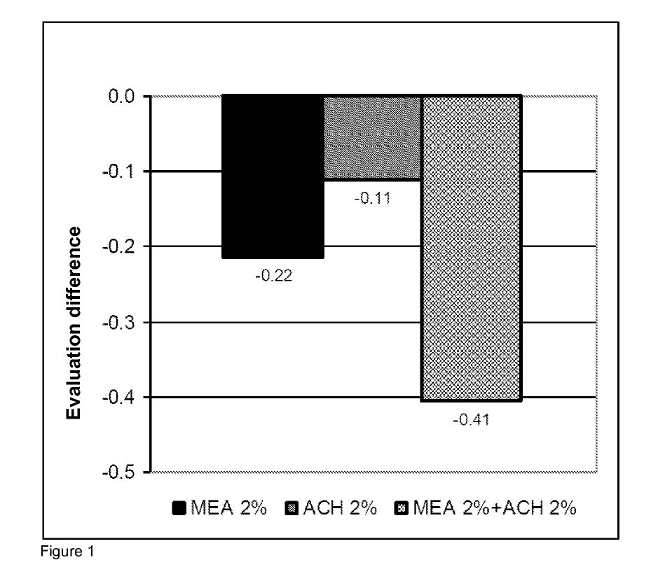

FIG. 1: effect on odour after six hours of the formulation according to the invention compared to individual components. In each case, the change relative to vehicle formulation is shown. The results for all test products were normalized beforehand by deducting the starting value (sweat odour without treatment).

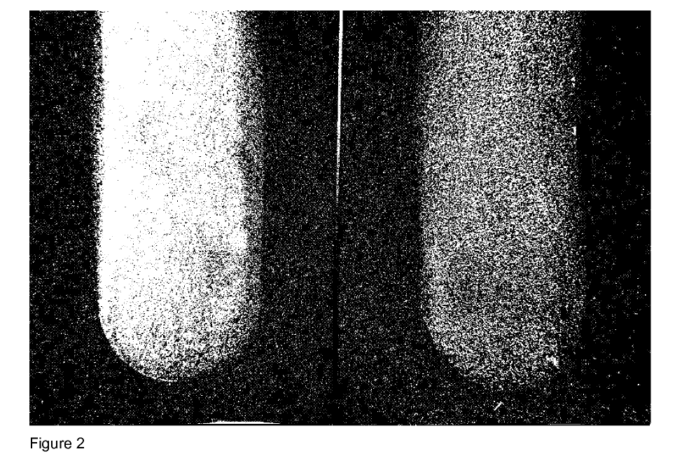

FIG. 2: photographic documentation of the whitening effect after converting the RGB colour image into a binary (black-white) image. Left: formulation A, right: formulation B.

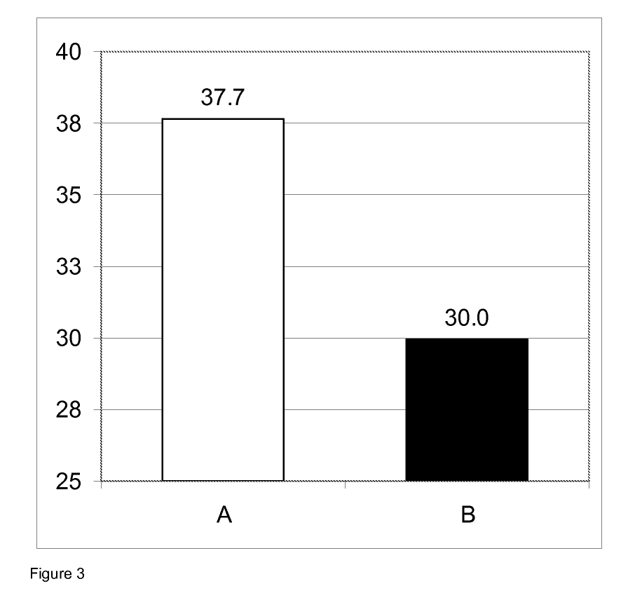

FIG. 3: parameter L* in the CIELAB colour space. On the left deodorant stick test formulation A, on the right deodorant stick test formulation B.

### EXAMPLE 1

### Effectiveness Testing of the Sweat-Odour-Inhibiting Effect

The study was carried out in the “Incomplete Block design”, randomized and double-blind. Two products were applied for each subject (half-side test) under the armpits in the form of a pump spray. Three professional sniffers carried out the evaluations. The group of subjects comprised 50 healthy volunteers, 33 female and 17 male aged from 20 to 65 years with a detectable body odour under the armpits. The subjects were committed to only wearing clothing made of pure cotton. The clothing, which was worn throughout the study, was not allowed to have been treated with perfumed detergents or fabric softeners. The study took place in the month of August.

During a preconditioning phase of ten days, the subjects were committed to using only a nonperfumed soap without antibacterial ingredients for cleaning their armpits. The cleaning of the armpits was carried out in accordance with a stipulated procedure for a period of 30 seconds.

During the main phase, the application of the test products was carried out after drying the armpits in a standardized form and with a defined amount of 5 pump strokes (ca. 0.7-0.8 g). Each subject applied two test products, one under the right armpit and one under the left armpit.

The evaluation was carried out during the main phase by three trained testers in each case six hours after the last washing operation (starting value) and six hours after a single application of the test products.

TABLE 1
Clear deodorant pump spray test formulations. Data in mass percent.
To produce the formulations, customary formulation processes known
to the person skilled in the art were used.

| | | | | | MEA 2% + |
|---|---|---|---|---|---|
| | Raw material | Vehicle | MEA 2% | ACH 2% | ACH 2% |
| A | TEGO ® Alkanol L 23 P (Laureth-23) | 2.0% | 2.0% | 2.0% | 2.0% |
| | TAGAT ® CH 40 (PEG-40 | 2.0% | 2.0% | 2.0% | 2.0% |
| | Hydrogenated Castor Oil) | | | | |
| | Water | 94% | 92% | 90% | 88% |
| | Propylene Glycol | 2.0% | 2.0% | 2.0% | 2.0% |
| B | Caprylic/capric acid ethanolamide | | 2.0% | | 2.0% |
| | (50% in Propylene Glycol) | | | | |
| | Aluminium Chlorohydrate (50% in | | | 2.0% | 2.0% |
| | Water) | | | | |
| C | Citric Acid (10% in Water) | pH 4.5 | pH 4.5 | pH 4.5 | pH 4.5 |

The degree of sweat odour was evaluated using a scale from zero to five (0=not perceptible, 1=very slight, 2=slight, 3=distinct, 4=strong, 5=very strong). Perceptible differences by comparison between the right and the left armpits were assessed with a difference of at least one value on the evaluation scale.

The olfactory assessment takes place six hours after a single product application. All evaluations were carried out in a specially climatized room, as a result of which a constant atmosphere with a room temperature of 22±1° C. and relative atmospheric humidity of 50±5% was ensured.

As the study showed, a reduction in the sweat odour compared with the vehicle formulation could be achieved after a single application of the test products. The decrease in sweat odour is lowest (−0.11) in the group which applied “ACH 2%”, whereas the decrease in sweat odour was more marked (−0.22) in the “MEA 2%” group. By combining identical concentrations of the two active ingredients, a reduction in sweat odour was observed in the “MEA 2%+ACH 2%” group which was more marked (−0.41) than one would have expected from the sum of the individual effects (−0.22+−0.11=−0.33). Consequently, by combining the two active ingredients “MEA 2%” and “ACH 2%”, an unforeseeable and surprising synergy effect was found.

### EXAMPLE 2

### Reduction in the Whitening of Aluminium Salt-Containing Deodorant Care Products

After applying deodorant care products, in particular aluminium salt-containing deodorant care products, very often white traces on skin and clothing are detected by the consumer. These white traces are mostly perceived as annoying and there are therefore efforts to reduce the whitening effect after applying deodorant care products. One approach here is the reduction of aluminium salt-containing components in the deodorant care formulation with a constant or even improved effectiveness of the product.

The aim was therefore to investigate whether by swapping aluminium salts with caprylic/capric acid ethanolamide in a deodorant stick formulation it is possible to reduce the formation of white traces after applying the deodorant stick, in particular on dark clothing.

For this purpose, two deodorant stick formulations were produced, the first containing 10% aluminium zirconium tetrachlorohydrex GLY, whereas in the second this amount was reduced to 5% and topped up with 5% caprylic/capric acid ethanolamide (Table 2).

TABLE 2
Deodorant stick test formulations. Data in mass percent. To produce
the formulations, customary formulation processes known to the
person skilled in the art were used.

| | Raw material | A | B |
|---|---|---|---|
| A | TEGO ® Alkanol 1618 (Cetearyl Alcohol) | 18.0 | 18.0 |
| | Hydrogenated Castor Oil | 2.5 | 2.5 |
| | TEGO ® Care 165 (Glyceryl Stearate; PEG-100 | 1.0 | 1.0 |
| | Stearate) | | |
| | Cyclomethicone | 51.5 | 51.5 |
| | TEGOSOFT ® MM (Myristyl Myristate) | 1.0 | 1.0 |
| | TEGOSOFT ® CR (Cetyl Ricinoleate) | 1.0 | 1.0 |
| B | TEGOSOFT ® PBE (PPG-14 Butyl Ether) | 10.0 | 10.0 |
| C | Talc | 3.0 | 3.0 |
| | Aerosil R 972 (Silica Dimethyl Silylate) | 1.0 | 1.0 |
| D | Aluminium Zirconium Tetrachlorohydrex GLY | 10.0 | 5.0 |
| E | Caprylic/capric acid ethanolamide | | 5.0 |
| F | Perfume | 1.0 | 1.0 |

In each case 0.2 g of the deodorant stick test formulations (Table 1) were applied alongside one another by two-fold spreading (back and forth) of the respective deodorant stick on a stretch of ca. 10 cm on black foam rubber. The photographic documentation of the whitening effect was carried out about six hours after application. Additionally, Skin-Colorimeter® CL 400 colour measurement probe and C+K Multi Probe Adapter System MPA (Courage+Khazaka Electronic GmbH) were used to determine the parameter L* in the CIELAB colour space. The parameter L* indicates the lightness and has the end points black (L=0) and white (L=100). The parameter L* was determined in each case by ten-fold measurement at various points of the deodorant stick test formulation applied to the foam rubber. The average values of these ten measurements are given in each case.

As the photographic documentation (FIG. 2) shows, the whitening effect after a two-fold spreading of the deodorant sticks was reduced if some of the aluminium salt in the deodorant stick test formulation is replaced with caprylic/capric acid ethanolamide.

Likewise, by measuring the lightness (L*) of the deodorant stick test formulations applied to the foam rubber, it could be shown that the formation of white deodorant traces is reduced (FIG. 3).

Consequently, a reduced whitening effect was achieved by replacing some of the aluminium salt with caprylic/capric acid ethanolamide.

### Example Formulations

Example Formulation PEG-Free Deodorant Roll-on

| Raw material | Percent by mass |
|---|---|
| TEGO ® Care PS (Methyl Glucose | 1.75 |
| Sesquistearate) | |
| TEGO ® Care PL 4 (Polyglyceryl-4 Laurate) | 0.25 |
| TEGOSOFT ® XC (Phenoxyethyl Caprylate) | 4.00 |
| TEGOSOFT ® E (PPG-15 Stearyl Ether) | 3.00 |
| Caprylic/capric acid ethanolamide | 2.00 |
| TEGODEO ® PY 88 (Zinc Ricinoleate) | 1.00 |
| Propylene Glycol | 2.00 |
| Water | ad 100 |
| Aluminium Chlorohydrate (50% in Water) | 15.00 |
| NATROSOL ® 250HHR (Hydroxyethylcellulose) | 0.75 |
| Microcare ® MEM (Dipropylene Glycol; | |
| Methylparaben; Ethylparaben; Aqua; | 0.80 |
| Methylisothiazolinone, Thor GmbH) | |
| Perfume | q.s. |

Example Formulation Antimicrobial Body Lotion

| Raw material | Percent by mass |
|---|---|
| TEGO ® Care PSC 3 (Polyglyceryl-3 | 2.50 |
| Dicitrate/Stearate) | |
| TEGO ® Alkanol 18 (Stearyl Alcohol) | 1.00 |
| TEGIN ® M Pellets (Glyceryl Stearate) | 1.50 |
| TEGOSOFT ® AC (Isoamyl Cocoate) | 5.00 |
| TEGOSOFT ® CT (Caprylic/Capric Triglyceride) | 5.00 |
| Caprylic/capric acid ethanolamide | 1.00 |
| Water | ad 100 |
| Glycerin | 3.00 |
| TEGO ® Carbomer 134 | 0.20 |
| TEGOSOFT ® XC (Phenoxyethyl Caprylate) | 2.00 |
| LACTIL ® (Sodium Lactate; Sodium PCA; Glycine; | 2.0 |
| Fructose; Urea; Niacinamide; Inositol; Sodium | |
| Benzoate; Lactic Acid) | |
| Preservative | q.s. |
| Perfume | q.s. |

Example Formulation Antiperspirant/Deodorant Stick

| Raw material | Percent by mass |
|---|---|
| TEGOSOFT ® APS (PPG-11 Stearyl Ether) | 5.00 |
| TEGOSOFT ® APM (PPG-3 Myristyl Ether) | 5.00 |
| TEGO ® Alkanol 18 (Stearyl Alcohol) | 16.25 |
| Hydrogenated Castor Oil | 1.75 |
| Cyclopentasiloxane (and) Cyclohexasiloxane | 42.00 |
| AEROSIL R 972 V (Silica Dimethylsilylate) | 3.00 |
| MICRO DRY ULTRAFINE (Aluminium | 20.00 |
| Chlorohydrate, Reheis) | |
| TEGO ® Cosmo P 813 (Polyglyceryl-3 Caprylate) | 0.50 |
| TEGODEO ® LYS (Zinc Ricinoleate; Lysine; | 4.00 |
| Propylene Glycol) | |
| Caprylic/capric acid ethanolamide | 2.50 |

Example Formulation W/O Antiperspirant Aerosol Spray

| Raw material | Percent by mass |
|---|---|
| ABIL ® EM 90 (Cetyl PEG/PPG-10/1 Dimethicone) | 2.00 |
| ISOLAN ® GPS (Polyglyceryl-4 Diisostearate/ | 2.00 |
| Polyhydroxystearate/Sebacate) | |
| TEGOSOFT ® TN (C12-15 Alkyl Benzoate) | 8.00 |
| Cyclopentasiloxane | 25.00 |
| Caprylic/capric acid ethanolamide | 2.00 |
| Perfume | 1.00 |
| Water | 34.20 |
| Aluminium Chlorohydrate (Reach 501, Reheis) | 20.00 |
| Propylene Glycol | 5.00 |
| Euxyl ® PE 9010 (Phenoxyethanol, | 0.80 |
| Ethylhexylglycerin; Schülke & Mayr GmbH) | |

Example Formulation Antiperspirant/Deodorant Aerosol Spray (Water-Free)

| Raw material | Percent by mass |
|---|---|
| Cyclopentasiloxane | 50.00 |
| Disteardimonium Hectorite | 3.00 |
| Perfume | 5.00 |
| Aerosil R 972 (Silica Dimethyl Silylate) | 2.00 |
| Aluminium Chlorohydrate | 25.00 |
| TEGOSOFT ® TN (C12-15 Alkyl Benzoate) | 8.00 |
| Caprylic/capric acid ethanolamide | 2.00 |
| Ethanol | 5.00 |

Example Formulation O/W Antiperspirant/Deodorant Roll-on

| Raw material | Percent by mass |
|---|---|
| TEGO ® Alkanol S 20 P (Steareth-20) | 1.00 |
| TEGO ® Alkanol S 2 Pellets (Steareth-2) | 2.20 |
| TEGOSOFT ® liquid (Cetearyl Ethylhexanoate) | 2.00 |
| TEGOSOFT ® APS (PPG-11 Stearyl Ether) | 2.00 |
| ABIL ® 350 (Dimethicone) | 0.50 |
| Caprylic/capric acid ethanolamide | 3.00 |
| Glycerin | 3.00 |
| Water | 52.30 |
| TEGODEO ® LYS (Zinc Ricinoleate; Lysine; | 4.00 |
| Propylene Glycol) | |
| Perfume | q.s. |
| Aluminium Chlorohydrate (Reach 501 Solution, | 30.00 |
| Reheis) | |
| Preservative | q.s. |

Example Formulation Deodorizing Wet Wipe Solution

| Raw material | Percent by mass |
|---|---|
| TEGO ® Betain F (Cocamidopropyl Betaine) | 2.00 |
| TEGO ® Betain 810 (Capryl/Capramidopropyl | 1.50 |
| Betaine) | |
| Propylene Glycol | 0.75 |
| REWODERM ® LI 63 (PEG-30 Glyceryl Cocoate) | 0.50 |
| Citric Acid Monohydrate | 0.50 |
| TEGODEO ® HY 77 (Zinc Ricinoleate; | 1.00 |
| Triethanolamine; Dipropylene Glycol; Lactic Acid) | |
| Caprylic/capric acid ethanolamide | 1.00 |
| Water | 92.65 |
| Tetrasodium EDTA | 0.10 |
| Citric Acid Monohydrate | pH 5.5 |
| Preservative | q.s. |
| Perfume | q.s. |

Example Formulation Deodorant Wipes Solution

| Raw material | Percent by mass |
|---|---|
| TAGAT ® CH 40 (PEG-40 Hydrogenated Castor Oil) | 3.0 |
| TEGO ® Alkanol L 23 P (Laureth-23) | 3.0 |
| TEGODEO ® LYS (Zinc Ricinoleate; Lysine; | 3.0 |
| Propylene Glycol) | |
| Caprylic/capric acid ethanolamide | 1.5 |
| Farnesol | 0.2 |
| Perfume | 0.5 |
| Water | 84.8 |
| Dipropylene Glycol | 4.0 |
| Citric Acid (50% in water) | q.s. |
| Preservative | q.s. |

Example Formulation Deodorant Foot Gel with Alcohol

| Raw material | Percent by mass |
|---|---|
| TEGODEO ® CW 90 (Zinc Ricinoleate; | 2.20 |
| Tetrahydroxypropyl Ethylenediamine; Laureth-3; | |
| Propylene Glycol) | |
| TEGO ® Alkanol L 4 (Laureth-4) | 4.40 |
| Alcohol | 32.35 |
| Neutrol TE (Tetrahydroxypropyl Ethylenediamine, | 3.75 |
| BASF SE) | |
| Lactic Acid (90%) | 0.20 |
| Menthol | 0.50 |
| Caprylic/capric acid ethanolamide | 2.00 |
| TEGO ® Carbomer 140 (Carbomer) | 1.50 |
| Water | 48.86 |
| Panthenol | 0.20 |
| Propylene Glycol | 2.00 |
| Colour | 2.04 |

Example Formulation Clear Hand Conditioning Gel

| Raw material | Percent by mass |
|---|---|
| ABIL ® EM 90 (Cetyl PEG/PPG-10/1 Dimethicone) | 2.00 |
| ABIL ® OSW 5 (Cyclopentasiloxane; | 2.00 |
| Dimethiconol) | |
| TEGOSOFT ® OP (Ethylhexyl Palmitate) | 4.00 |
| TEGOSOFT ® OS (Ethylhexyl Stearate) | 4.00 |
| Cyclopentasiloxane (and) Cyclohexasiloxane | 7.00 |
| TEGOSOFT ® CO (Cetyl Ethylhexanoate) | 2.00 |
| Perfume | q.s. |
| Caprylic/capric acid ethanolamide | 1.50 |
| Water | 14.40 |
| Magnesium Sulphate Heptahydrate | 0.80 |
| Propylene Glycol | 45.00 |
| Glycerin | 18.00 |
| Triclosan | 0.30 |
| Preservative | q.s. |

1. A formulation comprising:
at least one compound of formula (I) 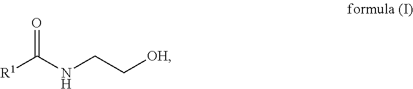
where
R¹=—C₇H₁₅ or —C₉H₁₉; and
an aluminium salt.

2. The formulation according to claim 1, wherein the aluminium salt is selected from aluminium acetate, aluminium behenate, aluminium benzoate, aluminium bromohydrate, aluminium butoxide, aluminium calcium sodium silicate, aluminium caprylate, aluminium capryloyl hydrolyzed collagen, aluminium chloride, aluminium chlorohydrate, aluminium chlorohydrex, aluminium chlorohydrex PEG, aluminium chlorohydrex PG, aluminium citrate, aluminium diacetate, aluminium dibenzoate/stearate hydroxide, aluminium dicetyl phosphate, aluminium dichlorohydrate, aluminium dichlorohydrex PEG, aluminium dichlorohydrex PG, aluminium dilinoleate, aluminium dimyristate, aluminium distearate, aluminium glycinate, aluminium hydrogenated tallow glutamate, aluminium hydroxy bis-methylene bis-di-t-butylphenyl phosphate, aluminium iron calcium magnesium germanium silicates, aluminium iron calcium magnesium zirconium silicates, aluminium iron silicates, aluminium isopropoxide, aluminium isostearate, aluminium isostearates/laurates/palmitates, aluminium isostearates/laurates/stearates, aluminium isostearates/myristates, aluminium isostearates/palmitates, aluminium isostearates/stearates, aluminium isostearyl glyceryl phosphate, aluminium laccate, aluminium lactate, aluminium lanolate, aluminium/magnesium hydroxide stearate, aluminium magnesium oxide, aluminium methionate, aluminium myristate, aluminium myristates/palmitates, aluminium PCA, aluminium phenolsulphonate, aluminium sesquichlorohydrate, aluminium sesquichlorohydrex PEG, aluminium sesquichlorohydrex PG, aluminium starch octenylsuccinate, aluminium stearate, aluminium stearates, aluminium stearoyl glutamate, aluminium sucrose octasulphate, aluminium sulphate, aluminium triformate, aluminium triphosphate, aluminium tristearate, aluminium undecylenoyl collagen amino acids, aluminium zinc oxide, aluminium zirconium trichlorohydrate, aluminium zirconium trichlorohydrex GLY, aluminium zirconium tetrachlorohydrate, aluminium zirconium tetrachlorohydrex GLY, aluminium zirconium tetrachlorohydrex PEG, aluminium zirconium tetrachlorohydrex PG, aluminium zirconium pentachlorohydrate, aluminium zirconium pentachlorohydrex GLY, aluminium zirconium octachlorohydrate, aluminium zirconium octachlorohydrex GLY, ammonium alum, ammonium silver zinc aluminium silicate, calcium aluminium borosilicate, cobalt aluminium oxide, magnesium/aluminium/hydroxide/carbonate, magnesium aluminium silicate, magnesium/aluminium/zinc/hydroxide/carbonate, potassium alum, potassium aluminium polyacrylate, silver magnesium aluminium phosphate, sodium alum, sodium aluminate, sodium aluminium chlorohydroxy lactate, sodium/aluminium hydroxide/oxalate/sulphate, sodium/aluminium/iron hydroxide/oxalate/sulphate, sodium/aluminium/iron/sulphate/citrate/hydroxide, sodium/aluminium/iron/sulphate/oxalate/hydroxide, sodium/aluminium/iron/sulphate/tartarate/hydroxide, sodium aluminium lactate, sodium phosphorus/zinc/calcium/silicon/aluminium/silver oxides, sodium potassium aluminium silicate, sodium silicoaluminate, sodium silver aluminium silicate, tromethamine magnesium aluminium silicate and alum.

3. The formulation according to claim 1, wherein the aluminium salt is aluminium chlorohydrate.

4. The formulation according to claim 1, further comprising less than 80% by weight of compounds of formula (II) 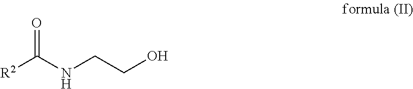
where
R²=an organic radical with a chain length of less than 7 or greater than 9,
based on the total amount of the compounds of the formula (I) and (II).

5. The formulation according to claim 1, wherein the at least one compound of formula (I) is present in an amount from 0.001 to 20% by weight, based on the total formulation.

6. The formulation according to claim 1, wherein the aluminium salt is present in amount from 0.01 to 50% by weight, based on the total formulation.

7. The formulation according claim 1, wherein the at least one compound of formula (I) includes a mixture of compounds of formula (I) wherein some of the compounds of formula (I) of the mixture have R₁=—C₇H₁₅ and other of the compounds of formula (I) of the mixture have R₁=—C₉H₁₉ and wherein a weight ratio of the compounds of formula (I) with R₁=—C₇H₁₅ to compounds of formula (I) with R₁=—C₉H₁₉ is in a range from 5:95 to 95:5.

8. The formulation according to claim 1, further comprising a deodorant active ingredient or an antiperspirant active ingredient.

9. The formulation accordingly to claim 1, further comprising at least one antimicrobial active ingredient.

10. The formulation according to claim 1, further comprising at least one foot care additive.

11. The formulation according to claim 1, further comprising at least one antiacne active ingredient.

12. The formulation according to claim 1, further comprising at least one antidandruff ingredient.

13. (canceled)

14. A method for reducing the growth of microorganisms, said method comprising:
applying a formulation comprising at least one compound of formula (I) 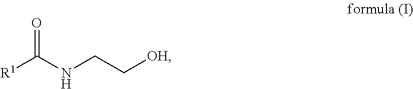
where
R¹=—C₇H₁₅ or −C₉H₁₉; and
an aluminium salt to an area in which a microorganism grows.

15. The method according to claim 14, wherein the microorganism is of the genera Microsporum, Epidermiphyton, Trichphyton or Gram-positive bacteria.

16. A method for reducing human body odor, said method comprising:
applying a formulation comprising at least one compound of formula (I) 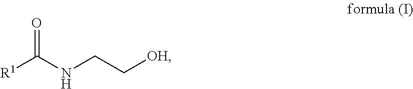
where
R¹=—C₇H₁₅ or —C₉H₁₉; and
an aluminium salt to human skin or to an article of manufacture.

17. (canceled)
